(19) United States
(12) Patent Application Publication
(10) Pub. No.: US 2014/0221232 A1
(43) Pub. Date: Aug. 7, 2014

(54) COMPUTER-IMPLEMENTED SYSTEM AND METHOD FOR THE PREDICTION OF CANCER RESPONSE TO GENOTOXIC CHEMOTHERAPY AND PERSONALISED NEOADJUVANT TREATMENTS (PCCP)

(71) Applicants: Jochen Prehn, Dublin (IE); Heinrich Huber, Kessel-Lo (BE); Markus Rehm, Dublin (IE)

(72) Inventors: Jochen Prehn, Dublin (IE); Heinrich Huber, Kessel-Lo (BE); Markus Rehm, Dublin (IE)

(21) Appl. No.: 14/129,727

(22) PCT Filed: Jun. 28, 2012
(86) PCT No.: PCT/EP2012/062566

§ 371 Date: Apr. 11, 2014

Related U.S. Application Data
(60) Provisional application No. 61/503,302, filed on Jun. 30, 2011.

(30) Foreign Application Priority Data
Jun. 30, 2011 (EP) 11172277.3

(51) Int. Cl. G06F 19/24; G01N 33/574

(52) U.S. Cl. CPC G06F 19/24; G01N 33/57496
USPC 506/9; 506/18; 702/19

(57) ABSTRACT

A method for predicting an individual's response to a treatment for cancer, the method comprising a step of assaying a biological sample from the individual to determine the abundance of a panel of two or more bio-markers comprising pro-apoptotic and/or anti-apoptotic biomarkers; inputting the abundance value for the two bio-markers into a computational model of a mitochondrial apoptosis pathway; and processing said abundance values using said computational model to provide a value to predict the individual's response to treatment.

### FIELD OF THE INVENTION

The invention relates to a computer-implemented system and method for predicting a patient's response to a treatment. In particular the invention relates to a method and system for predicting a patient's response to cancer treatment.

### BACKGROUND TO THE INVENTION

Solid cancer is, apart from cardiovascular diseases, the leading cause of death worldwide. Colorectal cancer is a leading cause of cancer with over an estimated 3.2 million new cases per year and 1.7 million deaths in 2008. The treatment of colorectal cancer patients depends on tumour location, disease stage and patient specific conditions. By standard care, stage 1 patients are treated by surgical resection; while a fraction of stage 2 patients receive additional radio-chemotherapy (chemo therapy and radiation). Stage 3 and stage 4 patients receive compulsory radio-chemotherapy. Treatment options have to be carefully balanced between expected benefit and potential drawback or risks. For cancer patients in stage 2, a decision has to be taken whether patients receive radio chemotherapy or not. In any stage where radio-chemotherapy is applied, the best suited treatment has to be chosen for the particular patient.

Conventional treatment for stage 2 (non metastatic) colorectal cancer is surgical resection with optional chemotherapy based on 5FU/oxaliplatin and leucovorin (FOLFOX) or 5FU/ironotecan (FOLFIRI). These drugs exert their beneficial activities by inducing DNA damage (‘genotoxic drugs’). For stage 3 and stage 4 colorectal cancer FOLFOX or FOLFIRI chemotherapeutic treatment is applied in the presence or absence of the angiogenic inhibitor bevazisumab (Avastin) or Cetuximab (kinase inhibition).

The benefit of chemotherapy in colorectal cancer is sometimes questionable. Stage 2 patients have an average 5 years survival of 70% which is only modestly increased by chemotherapy. The decision whether a stage 2 patient will receive chemotherapy or not is therefore a wager between the likelihood of benefit and of drawbacks and risks of treatment. This wager considers the patient's age, health condition, as well as a genetic and epigenetic fingerprint of tumour tissue. In turn, 5-year survival for stage 3 and stage 4 patients (50% vs. 20%) is low since patients either develop chemo-resistance or do not respond to chemotherapy at all in this stage. This poses the need for individualised treatment that is personalised to the patients genetic and tumour profile.

DNA damaging agents reduce tumour growth through cell cycle inhibition and induction of apoptosis. Resistance to DNA-damaging agents, the current standard therapeutics in the treatment of cancer, is one of the most important clinical problems in cancer treatment. It occurs in all common tumours such as colon cancer, breast cancer, to prostate cancer, and lung cancer. It is also of particular importance for the group of tumours that are intrinsically resistant and display cross-tolerance to multiple treatment paradigms, such as malignant glioma, melanoma, and pancreatic cancer. Tools that predict treatment responses and tools that direct the oncologists towards novel adjuvant treatment paradigms that enhance DNA damage-induced apoptosis are therefore urgently required.

While deciding upon treatment and finding the best treatment option is a multi factorial decision that needs to consider patient age and patient's general health, also genetic, epigenetic and proteomic profiles are taken into account today to evaluate cost/benefit of prospective treatments. Indeed, genotyping profiling or proteomic studies provide evidences that loss or perturbation of single gene/protein functions influences the clinical success of a certain treatment.

The following state of the art solutions are used to predict clinical success or to direct treatment options.

### Genetic Profiling:

Genotyping of somatic and germ line mutations are currently the most frequently used to direct clinical interventions. As an example, patients with mutations in the EGFR receptor gene will not receive the tyrosine kinase inhibitor Cetuximab that is used in metastatic colorectal cancer in addition to FOLFOX/FOLFIRI treatment.

### Epigenetic Profiling:

Epigenetic profiling relates to the determination of DNA methylation and histone acetylation profiles which are known to determine protein expression. During the last decade, epigenetic profiling became important as a tool for early cancer diagnoses. Similarly to genetic profiling, epigenetic profiling has potential to be used in treatment prediction and customisation by investigating what genes may be expressed or suppressed during treatment.

### Gene Expression Profiling:

Gene expression patterns using RNA Microarray may indicate what patterns are associated to a better treatment prognosis.

Chu et al (L. H. Chu and B. S. Chen, BMC Systems Biology 2008 2:56) provided a theoretical study of how intrinsic and extrinsic stress translates into a protein-interaction to network in cancer cells that governs the execution of apoptosis. The work described in Chu et al. does not allow for the use of quantitative information (neither by absolute protein levels nor by relative expressions of these biomarkers) when analysing a network response of biomarkers. The approach therefore falls short of investigating the effect of patient and cancer-specific quantitative protein fingerprints.

However, none of the practicing techniques have been proven to be accurate for predicting cancer cell death. It is an object of the present invention to overcome at least one of the above-mentioned problems.

### SUMMARY OF THE INVENTION

According to the present invention there is provided, as set out in the appended claims, a computer-implemented method for predicting an individual's response to a treatment for cancer, the method comprising a step of:
assaying a biological sample from the individual to determine the abundance of a panel of two or more biomarkers comprising pro-apoptotic and anti-apoptotic biomarkers;
inputting the abundance value for the two or more biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
processing said abundance values using said computational model to provide a value to predict the individual's response to treatment.

The invention predicts the response to chemotherapy, novel co-treatment regimes and/or novel adjuvant treatments from patient biopsies or tumour tissue samples. It provides a means of decision whether or not to give chemotherapy, for example, in the case of stage 2 colorectal cancer patients, or whether to administer novel co-treatments and/or novel adjuvant treatments. The invention provides a tool to predict how to optimise treatment for chemotherapy in advanced stages of tumour therapy (such as stage 3 and 4 colorectal cancer) and for the treatment of resistant cancers such malignant glioma. The invention also provides a tool to predict whether a patient, who was originally predicted not to respond to chemotherapy, will respond to novel adjuvant treatments or co-treatments that affect the mitochondrial pathway of apoptosis. The invention is designed to aid in clinical decision making. The invention can also be used to assess non-standard and experimental treatments for cancer acting on the mitochondrial apoptosis pathway. Such assessment may include pre-screening of the efficacy of putative drugs in advance of or during new clinical Phase II and III trials or prediction of individual patient response to such treatments before treatment administration. Novel adjuvant, non-standard and experimental treatments include proteasome inhibitors, Smac mimetic/IAP antagonists, or caspase-activating compounds.

Absolute protein levels can be obtained from biopsies, resected tumor material, or formalin-fixed, paraffin-embedded histopathology material using reverse phase protein arrays, quantitative Western Blotting, tissue microarray immunostaining or immunohistochemistry. This protein data will feed into the computational model that analyses protein-protein interaction.

The inventors realised that the dynamics of protein-protein networks (i.e. protein levels changing over time upon signal activation) is a non-linear function of cellular protein levels, and thus needs to be modelled as a non-linear function. In contrast, standard multivariate statistics approaches predict interactions in protein networks by using linear combinations of the protein levels of interest.

The technical field of the present invention is the provision of tools for predicting clinical outcome and suggesting co-therapies using biomarker expression levels as the basis for the predictive tool. The problem is how to combine different biomarkers that potentially indicate antagonising apoptosis function and arrange their predictive capacity in a quantitative way using a network of protein-protein interactions.

The prior art approach, for example as disclosed by Chu et al., does not provide a means to relate prediction from the protein-protein network to clinical success measured by means of patient survival, tumour regression or similar. The approach of Chu et al. to does not support dosage decisions of sensitisers to cell death (co-treatments) as proposed by the present invention.

The technical purpose of the present invention is to provide a combined predictivity output using two or more biomarkers selected from a set of biomarkers for apoptosis-susceptibility of different cancer cells. These cancer cells are from different patients and are characterised by their quantitative protein expressions of proteins involved in the mitochondrial pathway of apoptosis.

The computational model comprises a non-linear protein-protein network model of mitochondrial apoptosis pathway (referred to as the APOPTO-CELL), in which the abundance values for the proteins are inputted into the computational model. In one embodiment, the main output is the amount of substrate cleaved within a single cell as a consequence of caspase activation (percent of the cells, amounts of structural proteins and DNA that gets cleaved). On a cell population/tissue level, this amount translates to a likelihood of cell death.

The abundance values for the proteins are patient specific. The computational model uses these abundance values and calculates how the co-operating or antagonising influence of these proteins on each other results in cancer cell death/tumour shrinkage. Model output, using the non-linear protein-protein network, is the likelihood to what extent cancer cells of a tumour tissue with this specific protein abundance profile respond to chemotherapeutic stimuli and commit cell death. This likelihood is a value from 0 to 100% and is positively correlated with a favourable clinical response (A positive clinical response may be defined as tumour shrinkage after chemotherapy, no cancer relapse, or a patient with five year of survival).

There is a positive correlation between the likelihood predicted from the model and the likelihood of a positive clinical outcome. Examples are 1:1 relations of both likelihoods (70% cell death means 70% chance of clinical favourable outcome) and threshold to binary decisions (80% cell death indicates a positive outcome). Positive outcome is defined as above.

In one embodiment of the present invention, there is provided a computer-implemented method for predicting an individual's response to a treatment for cancer, the method comprising a step of:
assaying a biological sample from the individual to determine the abundance of two or more biomarkers from a panel comprising Apaf-1, procaspase-9, procaspase-3, XIAP, and Smac;
inputting the abundance value for the two or more biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
processing said abundance values using said computational model to provide a value to predict the individual's response to treatment.

In one embodiment of the present invention, there is provided a computer-implemented method for predicting an individual's response to a treatment for cancer, the method comprising a step of:
assaying a biological sample from the individual to determine the abundance of a panel of biomarkers;
inputting the abundance value for at least two of the panel of biomarkers into a computational model of a mitochondrial apoptosis pathway, wherein the pathway is activated through the permeabilisation of a cell's outer mitochondrial membrane by chemotherapeutically-induced stress; and
processing said abundance values using said computational model to provide a value to predict the individual's response to treatment.

In one embodiment, the processing step comprises inputting the abundance values into the computational model to provide a (caspase) apoptosis status, and correlating said status with a known response to provide said value to predict the individual's response to treatment.

In one embodiment, said processing step calculates resulting effector caspase activation to profile over time and compares the result with known results to provide said value to predict the individual's response to treatment.

In one embodiment, the value to predict the individual's response may be calculated using ordinary differential equations defined by

$$\frac{d {c}_{i}}{d t} = {\sum }_{j} {S}_{ij} * {v}_{j} + \frac{d {F}_{i}}{d t} , where \frac{d {c}_{i}}{d t}$$

represents the concentration change of molecule i over time, the velocity vⱼ is the reaction rate of reaction j, Sᵢⱼ denotes the stoichiometric matrix linking the reaction rates to the affected molecules, and

$$\frac{d {F}_{i}}{d t}$$

denotes a rate factor describing an external flux balance of the substance i.

In one embodiment, the Sᵢⱼ matrix describes the balance of substance j in equation i.

In one embodiment, the rate factor

$$\frac{d {F}_{i}}{d t}$$

links the input functions mimicking mitochondrial Smac release and Cyt-c release induced apoptosome formation to the model (F₁=F_{Apop}, F₁₄=F_{smac}, else Fᵢ=0).

In one embodiment, the reaction rates may be proportional to the product of the concentrations of the reacting substances.

In one embodiment, the panel of biomarkers may comprise Apaf-1, procaspase-9, procaspase-3, XIAP, and Smac.

In one embodiment, the abundance value for each biomarker is representative of protein levels for the sample.

In one embodiment of the present invention, the step of determining the abundance of the panel of biomarkers may further comprise obtaining protein profiles by any one or more of: tissue microarray immunostaining, immunohistochemistry, reverse phase protein array analysis, or quantitative Western blot.

In one embodiment of the present invention, the abundance value for each biomarker may be compared with a reference abundance value for each biomarker from quantitative Western blotting. The reference abundance value for each biomarker is collated from protein abundance values from samples obtained from a cohort of patients exhibiting the same type of cancer and at the same stage of cancer progression. An average abundance value for each biomarker may also be obtained from a databank of average abundance values from patients having various stages of cancer, for example colorectal cancer. In the present invention, the average abundance values of the patient group described herein were found to be 0.083+/−0.006 μM for procaspase-3, 0.006 μM+/−0.010 for procaspase-9, 0.102+/−0.067 μM for XIAP, 0.548+/−0.548 μM for Smac, and 0.407+/−0.407 μM for Apaf-1. When an average abundance value is available, such values may be inputted into the computer-implemented system to determine whether a patient would respond to chemotherapeutic treatments or whether the patient would respond to novel co-treatment regimes or to novel adjuvant treatments for solid tumours, such as proteasome inhibitors, Smac mimetic/IAP antagonists, or caspase-activating compounds with or without chemotherapeutic agents.

In one embodiment of the present invention, the cancer may be selected from the group consisting of myeloma, prostate cancer, glioblastoma, lymphoma, fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; to acoustic neuroma; oligodendroglioma; meningioma; melanoma; retinoblastoma; and leukemias.

In one embodiment of the present invention, the panel of biomarkers may comprise Smac and Apaf-1, and optionally one or more biomarkers selected from procaspase-9, procaspase-3 and XIAP.

In one embodiment of the present invention, the panel of biomarkers may comprise one or more biomarkers selected from procaspase-9, procaspase-3 and XIAP.

In one embodiment of the present invention, the panel of biomarkers may comprise Apaf-1 and one or more biomarkers selected from procaspase-9, procaspase-3 and XIAP.

In one embodiment of the present invention, the panel of biomarkers may comprise Smac and one or more biomarkers selected from procaspase-9, procaspase-3 and XIAP.

In one embodiment of the present invention, there is provided a computer-implemented method for predicting an individual's response to a treatment for cancer, the method comprising a step of:
assaying a biological sample from the individual to determine the abundance of a panel of two or more biomarkers comprising pro-apoptotic and anti-apoptotic biomarkers;
inputting the abundance value for the two or more biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
processing said abundance values using said computational model to provide a value to predict the individual's response to treatment.

In a further embodiment of the present invention, there is provided a computer-implemented system for predicting an individual's response to a treatment for cancer, the system comprising:

means for assaying a biological sample from the individual to determine the abundance of a panel of two or more biomarkers comprising pro-apoptotic and anti-apoptotic biomarkers;
means for inputting the abundance value for the two or more biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
means for processing said abundance values using said computational model to provide a value to predict the individual's response to treatment.

In one embodiment, the processing comprises inputting the abundance values into the computational model to provide a (caspase) apoptosis status, and correlating said status with a known response to provide said value to predict the individual's response to treatment.

In one embodiment, said processing means may calculate resulting effector caspase activation profile over time and may compare the result with known results to provide said value to predict the individual's response to treatment.

In one embodiment, the panel of biomarkers may be selected from Apaf-1, procaspase-9, procaspase-3, XIAP and Smac.

In one embodiment of the present invention, there is provided a computer-implemented system for predicting an individual's response to a treatment for cancer, the system comprising:
means for assaying a biological sample from the individual to determine the abundance of a panel of pro-apoptotic and anti-apoptotic biomarkers;
means for inputting the abundance value for two or more of the biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
means for processing said abundance values using said computational model to provide a value to predict the individual's response to treatment.

In one embodiment of the present invention, there is provided a computer program comprising program instructions for causing a computer to perform the method as described above.

In one embodiment of the present invention, there is provided a method of identifying an individual having cancer who is suited for treatment with a chemotherapeutic agent, which method employs a step of identifying an individual who will respond to a treatment according to the method as described above, wherein the individual identified is treated with the chemotherapeutic agent.

Generally speaking, the biomarker is a protein. However, the computer-implemented method of the invention may also be performed by detecting differential expression by other means, for example, the enumeration of mRNA copy number.

Generally speaking, the biomarker is a component of the mitochondrial apoptosis pathway, for example, those listed in Table 1. In the specification, the phrase “a mitochondrial apoptosis pathway” should be understood to mean the activation of the apoptotic pathway in a cell through the permeabilisation of the outer mitochondrial membrane (mitochondrial outer membrane permeabilisation (MOMP)) caused by a range of stress stimuli such as UV radiation, gamma radiation, heat, viral virulence factors, growth-factor deprivation, DNA-damaging agents (such as chemotherapeutic agents fluorouracil (5FU), oxaliplatin, ironotecan, etoposide, cisplatin, and doxorubicin), receptor kinase inhibitors (such as chemotherapeutic agents cetuximab, 5-fluorouracil, oxaliplatin, leucovorin, irinotecan, and bevazisumab) and the activation of some oncogenic factors. Once cells have undergone MOMP, the downstream part of the mitochondrial apoptosis pathway is executed. This pathway is governed by a cascade of enzymatic reactions. By this cascade, the death signal is balanced against protective mechanisms. Protective mechanisms involve protective enzymes that bind and to deactivate enzymes that mediate cell death. The interplay of proteins that induce cell death and such that exert protection is executed by control feedback and feed forward steps. The cascade downstream to MOMP is depicted and explained in FIG. 1.

Generally speaking, the biological sample is a blood sample, especially blood serum or plasma. However, other biological samples may also be employed, for example, cerebrospinal fluid, saliva, urine, lymphatic fluid, or cell or tissue extracts.

Generally speaking, the individual is a human, although the computer-implemented method of the invention is applicable to other higher mammals.

In this specification, the term “cancer” should be understood to mean a cancer that is treated by chemotherapeutic regimens. An example of such a cancer include multiple myeloma, prostate cancer, glioblastoma, lymphoma, fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

In the specification, the term “positive outcome” should be understood to mean a patient who responds positively to chemotherapeutic treatment, while the term “negative outcome” should be understood to mean a patient who does not respond to chemotherapeutic treatment.

In the specification, there term “treatment” should be understood to mean standard to chemotherapeutic treatment, novel co-treatment regimes and/or novel adjuvant treatments (including non-standard/experimental treatments). Chemotherapeutic treatments are those using compounds such as for example chemotherapeutic agents fluorouracil (5FU), oxaliplatin, irinotecan, etoposide, cisplatin, doxorubicin, and receptor kinase inhibitors such as cetuximab and sorafenib. As an example, conventional treatment for stage 2 (non metastatic) colorectal cancer is surgical resection with optional chemotherapy based on 5FU/oxaliplatin and leucovorin (FOLFOX) or 5FU/ironotecan (FOLFIRI). These drugs exert their beneficial activities by inducing DNA damage (‘genotoxic drugs’). For stage 3 and stage 4 colorectal cancer FOLFOX or FOLFIRI chemotherapeutic treatment is applied in the presence or absence of the angiogenesis inhibitor bevacizumab (Avastin) or the HER2 inhibitor Cetuximab (kinase inhibition). Novel adjuvant, non-standard and experimental treatments include proteasome inhibitors, Smac mimetic/IAP antagonists, or caspase-activating compounds. The treatment using the above indications can invoke permeabilisation of the cancer cell's outer mitochondrial membrane by chemotherapeutically-induced stress.

In the specification, the term “pro-apoptotic biomarker” should be understood to mean a molecule involved in promoting and/or progressing the process of programmed cell death (biochemical events leading to characteristic cell changes including blebbing, cell shrinkage, nuclear fragmentation, chromatin condensation, and chromosomal DNA fragmentation) and cell death. Pro-apoptotic biomarkers can be selected from the group comprising APAF-1, Smac, procaspase-9, procaspase-3, Pro-caspase-7, OMI/HtrA2, cytochrome-C, Procaspase-2, Procaspase-6, CAD (Caspase-activated DNAse), and PARP-1.

In the specification, the term “anti-apoptotic biomarker” should be understood to mean a molecule involved in preventing and/or stopping the process of programmed cell death.

Anti-apoptotic biomarkers can be selected from the group comprising XIAP, cIAP1, cIAP2, Survivin, Aven, Hsp70, and Hsp90.

There is also provided a computer program comprising program instructions for causing to a computer program to carry out the above method which may be embodied on a record medium, carrier signal or read-only memory.

### BRIEF DESCRIPTION OF THE DRAWINGS

The invention will be more clearly understood from the following description of an embodiment thereof, given by way of example only, with reference to the accompanying drawings, in which:—

FIG. 1 illustrates an apoptosis pathway following mitochondrial permeabilisation in response to DNA damage. Following permeabilisation of the mitochondrial outer membrane cytochrome-c and SMAC (dots) are released into the cytosol, initiating the apoptosis execution network modeled by Ordinary Differential Equations (ODEs). As output, the model calculates caspase-dependent substrate cleavage over time.

FIG. 2 illustrates a strategy for comparison of in silico predictions of tumor cell death (based on protein profiles) with clinical outcome.

FIG. 3 is flow chart illustrating the method and system of the present invention from obtaining a sample, quantitating protein levels, predicting treatment outcome using the APOPTO-CELL Tool of the present invention.

FIG. 4 illustrates (A) Quantitative Western Blot analysis of five pro-apoptotic or anti-apoptotic proteins in colorectal Dukes' B (Stage II CRC—13 patients) and Dukes C (Stage III CRC—17 patients) samples. (B) Apaf-1, (C) XIAP, (D) pro-caspase 9, (E) pro-caspase 3 and (F) Smac.

FIG. 5 illustrates screen-grabs from the interface of the APOPTO-CELL Tool of the present invention used for therapy prediction. (A-B) Tool predicts a positive response to chemotherapeutic stimuli. (A) Graphical user interface of the APOPTO-CELL-tool. Data input panels ‘Mode 0’ and ‘General parameter’ (first three data entries) contain the input for the up to five proteins that are used in the tool. Other parameters are technical parameters that depend on the implementation. Not all five protein concentrations are needed and default protein concentration can be used. Default protein concentrations were obtained from cumulated patient data (such as those illustrated in FIG. 4) and found to be 83 nM for procaspase-3, 6 nM for procaspase-9, 102 nM for XIAP, 548 nM for Smac, and 407 nM for Apaf-1. (B) Model output predicting full response to chemotherapy indicated by 100% cancer cell death. (C-D) The APOPTO-CELL tool predicts a negative response to chemotherapeutic stimuli. (C) The amount of the anti-apoptotic protein XIAP was increased and the calculation was restarted. (D) APOPTO-CELL predicts non response to chemotherapy indicated by the flat line.

FIG. 6 illustrates that when the protein concentrations obtained from the results in FIG. 4 are inputted into the computer-implemented method of the present invention, as illustrated in FIG. 5, the method predicts apoptosis impairment progressing with disease stage, for example, in normal tissue there is a prediction of 100% progression to apoptosis, a 69% probability of progression to apoptosis for Dukes' B stage patients analysed, and a 58% likelihood of progression to apoptosis for Dukes' C stage patients analysed.

FIG. 7 illustrates a validation of the computer system of the present invention of mitochondrial apoptosis “APOPTO-CELL” in a clinical setting of patients treated for colorectal cancer. The study demonstrated the suitability of the system for predicting treatment response. In silico predictions compared with patients having positive [Upper panel: 12 patients] and negative [Lower panel: 8 patients] response to chemotherapy are shown.

FIG. 8 illustrates that the APOPTO-CELL-tool predicts correlation between the likelihood of cancer cells undergoing apoptosis with survival for patients suffering from glioblastoma multiformae (GBM). (A-F) Protein quantifications from tumour samples of GBM patients (A) were quantified by quantitative Western Blotting. Protein levels of the Apopto-Cell proteins Procaspase-3 (B), APAF-1 (C), Procaspase-9 (D), Smac (E), and XIAP (F) were quantified. (G-H) Prediction of the susceptibility of cancer cells undergoing cell death (apoptosis) correlates with duration of patient survival. 20 Patients were grouped into either (G) Long term or (H) short term survivors according their clinical outcome. Patients that survived longer were also predicted to have tumours that are more susceptible to cancer cell death indicated by shorter response time to treatment (curves reach 100% more quickly). Each curve indicates one patient.

FIG. 9 illustrates predicting the effects of alternative, targeted treatment strategies in colorectal cancer patients. The model of the present invention was expanded to include the effects of XIAP antagonists, proteasome inhibition, and direct activation of pro-Caspase 3. The effects of these drugs on caspase activation profiles in to patients with less than 25% substrate cleavage predicted. Panels represent individual patients.

### DETAILED DESCRIPTION OF THE DRAWINGS

### Protein Profiles

Protein profiles are obtained either by tissue microarray immuno-staining, immunohistochemistry, reverse phase protein array analysis, or by quantitative Western Blot. For Western blot, quantitative protein levels are obtained by comparison against purified proteins of known concentrations or against cell extracts with known protein quantities. Test data sets permits the calibration of tissue microarray profiles, immunohistochemistry profiles, or reverse phase protein arrays against protein profiles obtained from quantitative Western blot analysis.

### Computer System

DNA damaging agents induce tumor regression through the activation of apoptosis. The computer system uses processing means to translate a chemical pathway of apoptosis into a mathematical set of equations that describe protein-protein interactions.

The reaction network describes the process of effector caspase activation subsequent to mitochondrial outer membrane permeabilisation. Chemotherapeutic treatment is simulated by invoking mitochondrial outer membrane permeabilisation as an initiator signal which then manipulates network targets. Initiator signals assume active proteins while network targets are manipulated by protein binding.

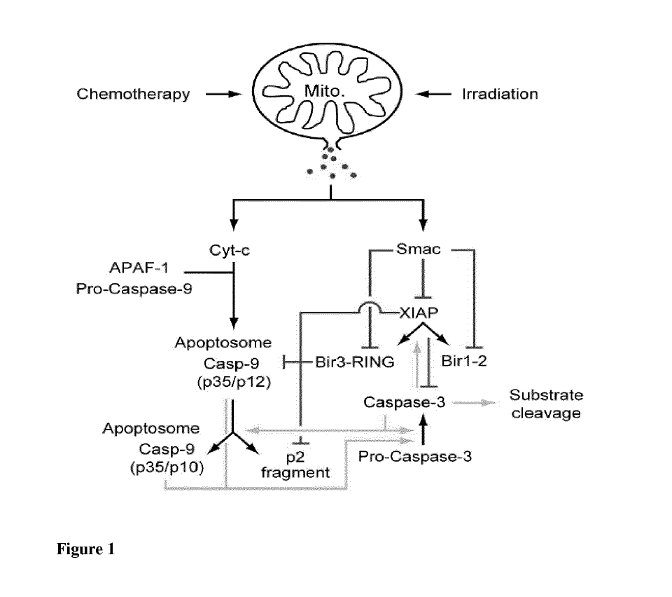

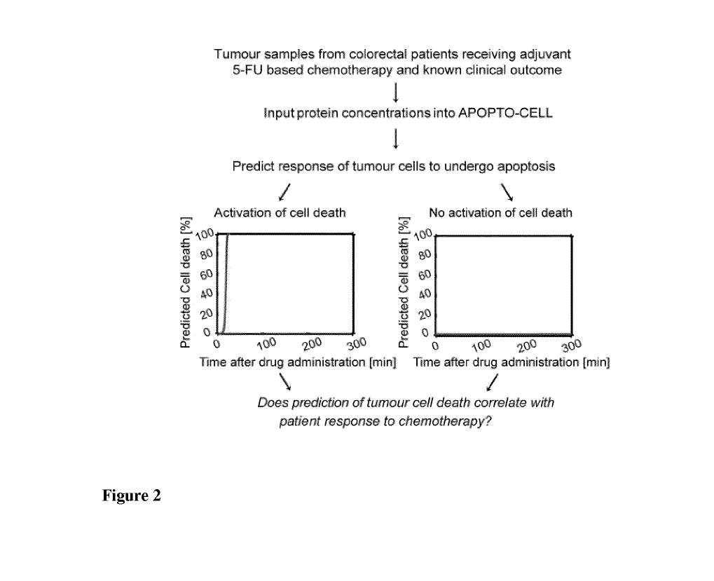

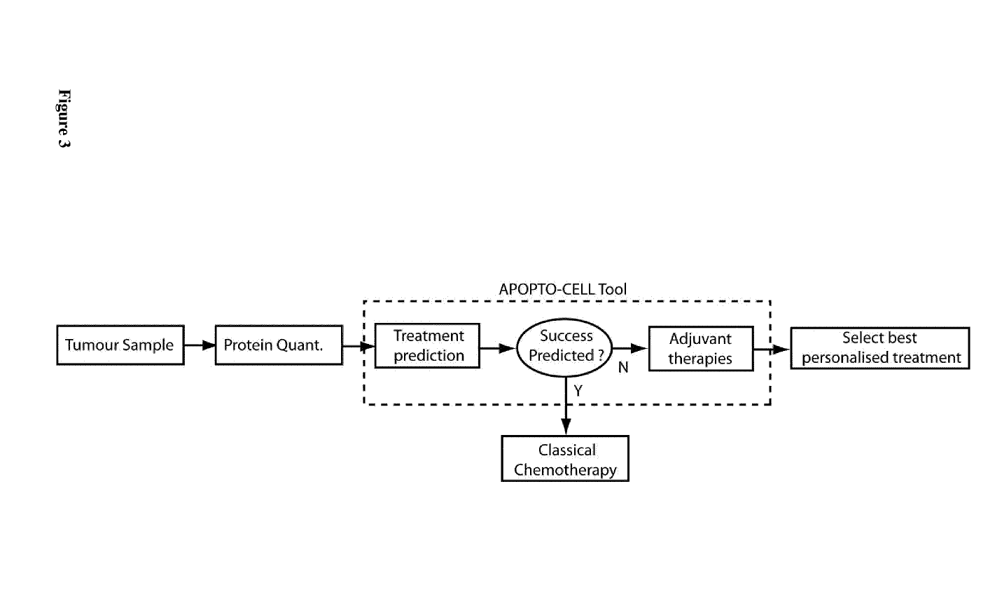

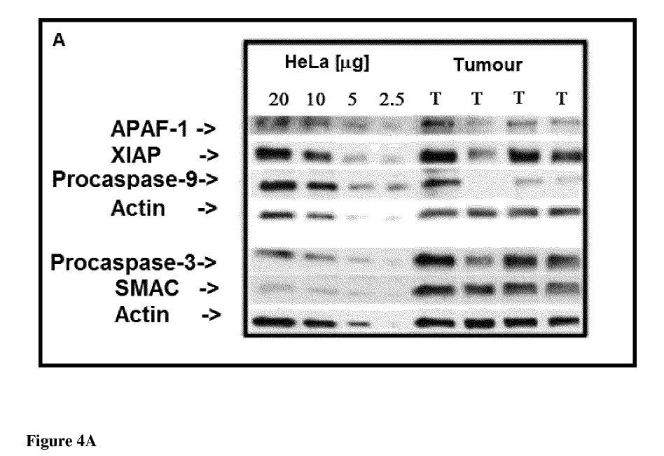

The modelled signalling network is depicted in FIG. 1. Model input and output, biological background, mathematical modelling of the protein-protein interaction and inclusion of therapies are described below. Abbreviations used in the modelling syntax are explained in Table 1. Initial conditions of the system and input parameters are defined in Table 2. The modelled reactions are listed in Table 3, the respective kinetic constants and binding constants are specified in Table 4. Protein interactions and enzymatic cleavage reactions were modelled following mass action kinetics. Based on ordinary differential equations (ODEs), the model was implemented in MATLAB (The Mathworks, UK) and was solved numerically by a four step Runge-Kutta Gear (Gear CW 1971) integration. The model is referred to herein as the APOPTO-CELL.

### Model Input and Output

Absolute protein concentrations of procaspase-3, procaspase-9, XIAP, SMAC, APAF-1 and the assumption of mitochondrial outer membrane permeabilisation (MOMP) are input.

MOMP is assumed to be initiated by two input functions mimicking mitochondrial Smac release (Equation 1) and Cyt-c release induced apoptosome formation (Equation 1 and 2):

F_{Smac}(t)=Smac^{tot}*[1−exp(t/t_{Smac,1/2} log(2)] Equation 1

F_{Apop}(t)=Apop^{tot}*[1−exp(t/t_{Apop,1/2} log(2)] Equation 2

Smac and Cyt-c release kinetics from mitochondria were described by the protein accumulation in the cytosol, with the half time of the release being t_{Smac,1/2} and t_{Cyt-c,1/2}, respectively. The Cyt-c initiated apoptosome formation encompasses the complexity of Apaf-1 oligomerization and recruitment of procaspase-9. Its kinetic parameter (t_{Apop,1/2}) closely resembles the Cyt-c release kinetic (Goldstein J C, W. N., Juin P, Evan G I and Green D R (2000). “The coordinate release of cytochrome c during apoptosis is rapid, complete and kinetically invariant.” Nature Cell Biology 2.; Hill, M. M., C. Adrain, et al. (2004). “Analysis of the composition, assembly kinetics and activity of native Apaf-1 apoptosomes.” Embo J 23(10): 2134-2145.; Twiddy, D., D. G. Brown, et al. (2004). “Pro-apoptotic proteins released from the mitochondria regulate the protein composition and caspase-processing activity of the native Apaf-1/caspase-9 apoptosome complex.” J Biol Chem 279(19): 19665-19682) and is remodelled by an analytical function describing the resulting amount of procaspase-9 recruited to the apoptosome.

Model output was caspase-3 dependent substrate cleavage over time.

The release of Cytochrome-c (Cyt-c) and Smac was modelled to be caspase-independent, and kinetically based on their individual release kinetics. Omi/HtrA2, another protein released from mitochondria with a function similar to Smac, was neglected as an independent parameter. The release of Cyt-c triggers the formation of the apoptosome, and Cyt-c was modelled not to restrict apoptosome formation. The kinetics of apoptosome formation was re-modelled from previously published data, and in HeLa cells is stoichiometrically limited by the total amount of procaspase-9. As in other cell types where Apaf-1 may restrict apoptosome formation, the model was designed to adjust the amount of apoptosome formation to the respective limiting protein fraction (procaspase-9 or Apaf-1).

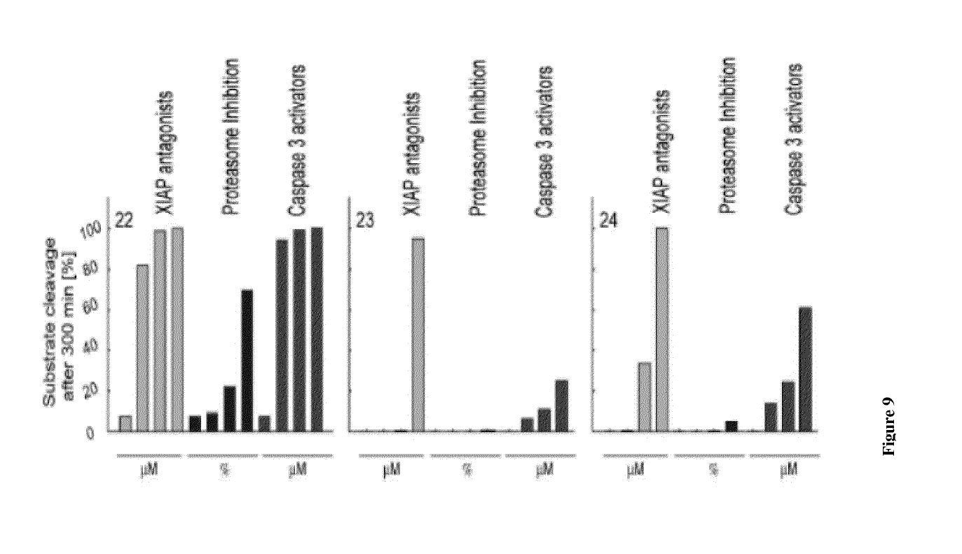

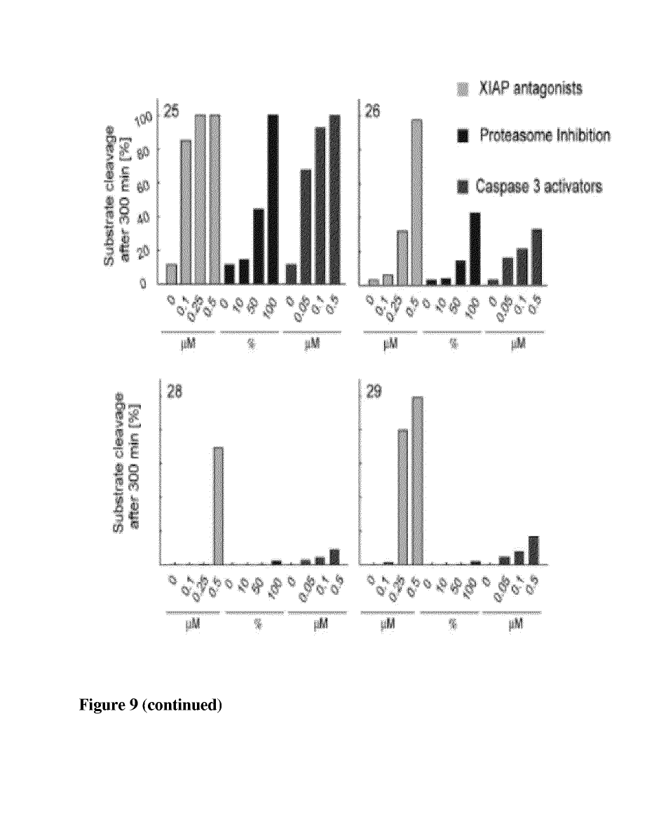

From these inputs, the model calculated the resulting effector caspase activation profile over time, taking the following into account: Apoptosome-bound procaspase-9 exists in an active conformation, is able to auto-catalytically process itself to its p35/p12 form and activates effector caspases-3 and -7. In a positive feedback loop, active caspase-3 processes caspase-9 to its p35/p10 form, resulting in an increased caspase-9 activity. XIAP was modelled to inhibit active caspase-3, caspase-7, and p35/p12 caspase-9, but not the fully processed p35/10 caspase-9. Furthermore, active caspase-3 can cleave XIAP into its BIR1-2 and BIR3-RING fragments. IAP family members cIAP-1 and -2 in comparison to XIAP have lower affinity for caspases, are less stable, and were therefore neglected. Other regulators of caspase activity such as transcriptional regulation or phosphorylation were neglected as independent parameters. Effects of such mechanisms can be modelled as altered amounts of caspase-3 or apoptosome bound active caspase-9 in the system such as performed in FIG. 9.

Proteins binding to XIAP were modelled to be subsequently ubiquitinated and degraded by the proteasome. Although effector caspase activation impairs protein synthesis and protein degradation these effects only mildly influenced the signalling network at later time points.

### Modelling of the Protein-Protein Interaction Network

Once initiated, the model calculates the response of the system and allows the following of all proteins and complexes over time. Protein concentrations (c₁, c₂, . . . , cₘ) were considered to be numerically continuous and concentration gradients were neglected (one compartment model). The reaction rates are dependent on the protein concentrations and on the kinetic constants (k^{(on)}₁, k^{(on)}₂, . . . , k^{(on)}ₙ, k^{(Off)}₁, k^{(off)}₂, . . . , k^{(off)}ₙ) for forward (on) and backward (off) reactions, respectively. Temporal protein profiles were calculated with a system of ODEs generated from linear combinations of the reaction rates:

$$\frac{d {c}_{i}}{d t} = {\sum }_{j} {S}_{ij} * {v}_{j} + \frac{d {F}_{i}}{d t} \\ where \frac{d {c}_{i}}{d t} \qquad Equation 3$$

represents the concentration change of molecule i over time. The velocity vⱼ is the reaction rate of reaction j, and Sᵢⱼ denotes the stoichiometric matrix linking the reaction rates to the affected molecules. Reaction rates are proportional to the product of the concentrations of the reacting substances. The Sᵢⱼ matrix describes the balance of substance j in equation i. Reaction rates as well as the stoichiometric matrix were generated from the list of reactions (Table 3). Finally, the rate factor

$$\frac{d {F}_{i}}{d t}$$

describes an external flux balance of the substance i and thereby links the input functions to the model (F₁=F_{Apop}, F₁₄=F_{Smac}, else Fᵢ=0).

Conventional approaches of the prior art in determining or predicting the clinical success of or to direct treatment options for a patient, such as principal component or discriminant analysis, use linear combinations of gene or protein expression levels. The approach of the present invention, however, focuses on studying the molecular interaction of proteins and the topology of their interaction based on protein interaction kinetics. Such chemical protein interactions kinetics are based on non-linear dynamics such as mass action, Michaelis-Menten or Hill kinetics. The use of linear combinations in the calculations of the prior art approaches is unable to use such non-linear dynamics.

Therefore, only ODEs can correctly integrate the molecular interaction of several proteins that in combination may act as predictive markers. Combining biomarkers based on the molecular protein interaction and the topology of the interaction network is therefore an unprecedented approach in biomarker discovery.

Implementation of Co-Treatment Regimes to Concomitant adjuvant treatments concomitant to 5FU/Oxaliplatin were investigated and include Smac-mimetics, proteasome inhibitors, or procaspase-3/6/8 activating compounds, at different dosages. The effect of Smac-mimetics was included in the model to assume additional levels of Smac proteins with initial cytosolic concentrations of 10, 25, and 500 μM and mimicking the entire behaviour of Smac, including its binding to XIAP and its own proteasomal degradation. Proteasome-inhibition was modelled to reduce the degradation rate of all activated proteins by a factor 0.9 (10% inhibition), 0.5 (50% inhibition), or 0 (100% inhibition). Procaspase activating compounds were initially present with cytosolic concentrations of 10, 25, and 500 μM and assumed to enzymatically activate procaspase-3 by a mass action kinetics of k^{cat}=0.068 (uM min)⁻¹.

In FIG. 9 the modelled treatment response is given for 7 patients (denoted by ‘22’ to ‘29’). Each patient was assumed to be subjected to the three co-treatment regimes, ‘XIAP antagonists’, ‘Proteasome inhibitors’ and ‘Caspase 3 activators’ that were supposed to be administered in addition to classical FOLFOX and FOLFIRI chemotherapy. Each co-treatment was modelled to be given at different doses (0.1, 0.25, 0.5 μM). All calculations were performed to a time point of 300 minutes after MOMP and substrate cleavage indicating cancer cell death was obtained as output. High amounts of substrate cleavage were considered to mimic high cancer cell death and thus were assumed to indicate a positive treatment response. As comparison predicted response to FOLFOX or FOLFIRI treatment is given (bars with doses marked with ‘0’). As can be seen, patient ‘25’ (and to a minor extent also patient ‘22’) is predicted to respond to chemotherapy of FOLFOX or FOLFIRI in addition with any of the three co-treatments. In contrast, patients ‘28’ and ‘29’ were only predicted to respond to FOLFOX or FOLFIRI in combination with high doses of ‘XIAP antagonists’.

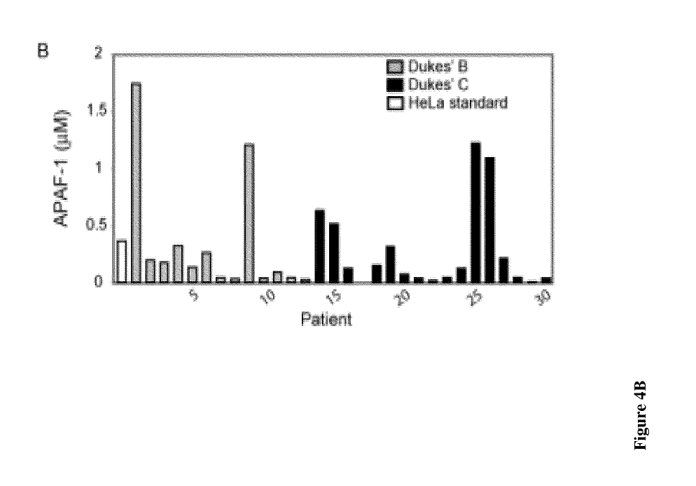

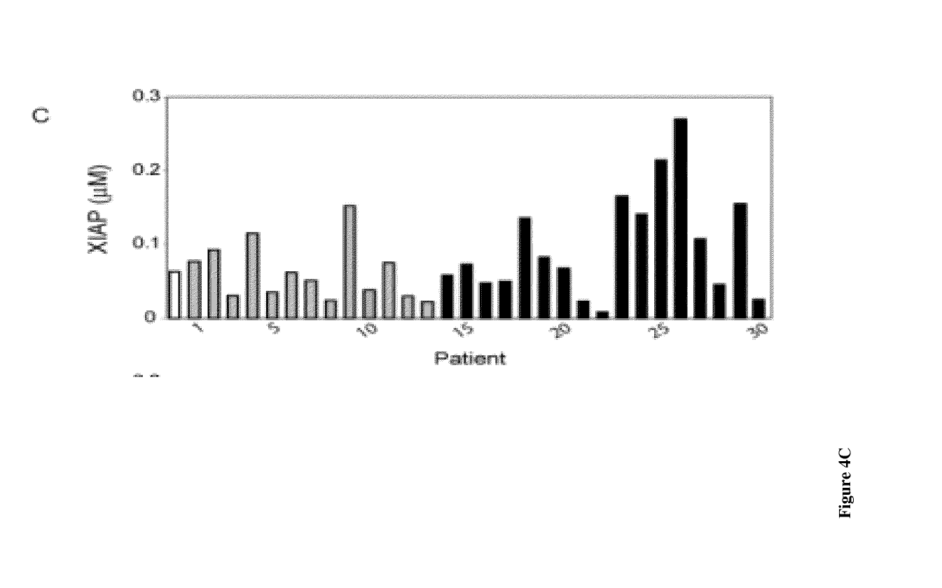

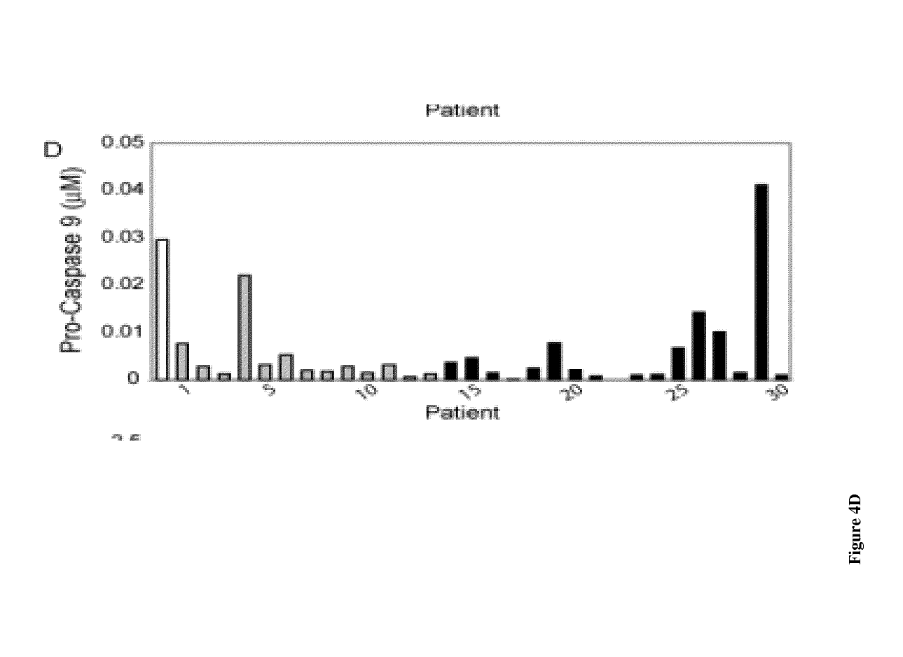

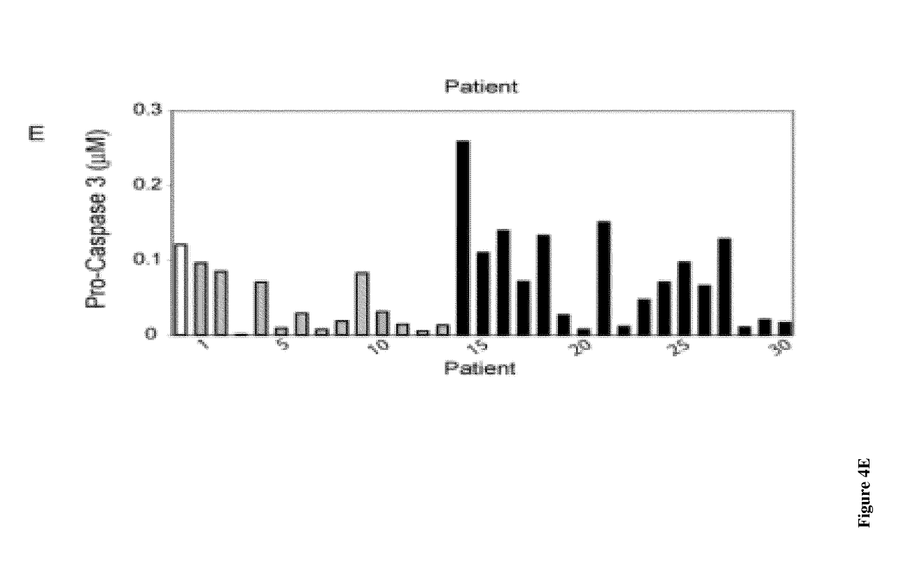

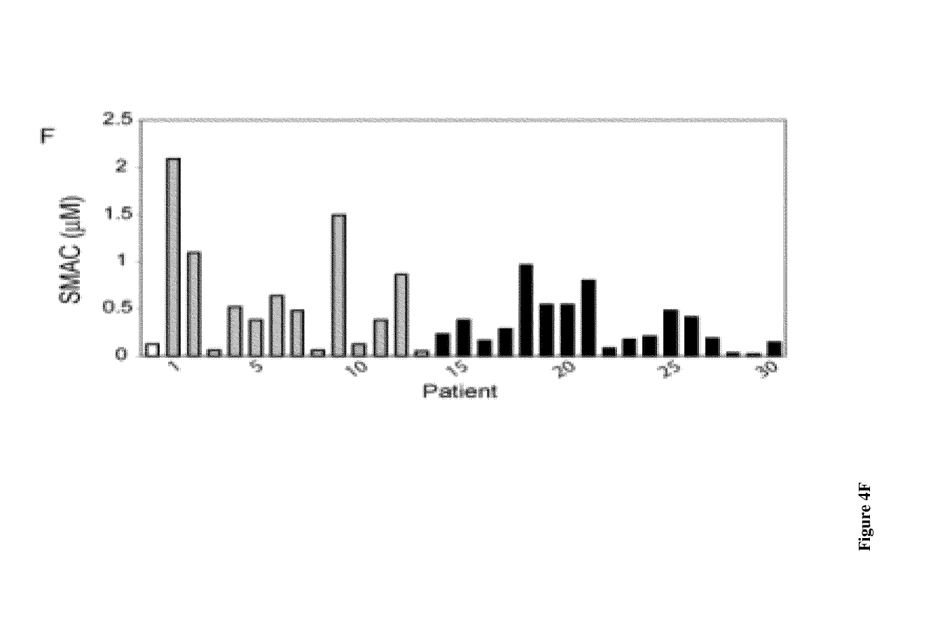

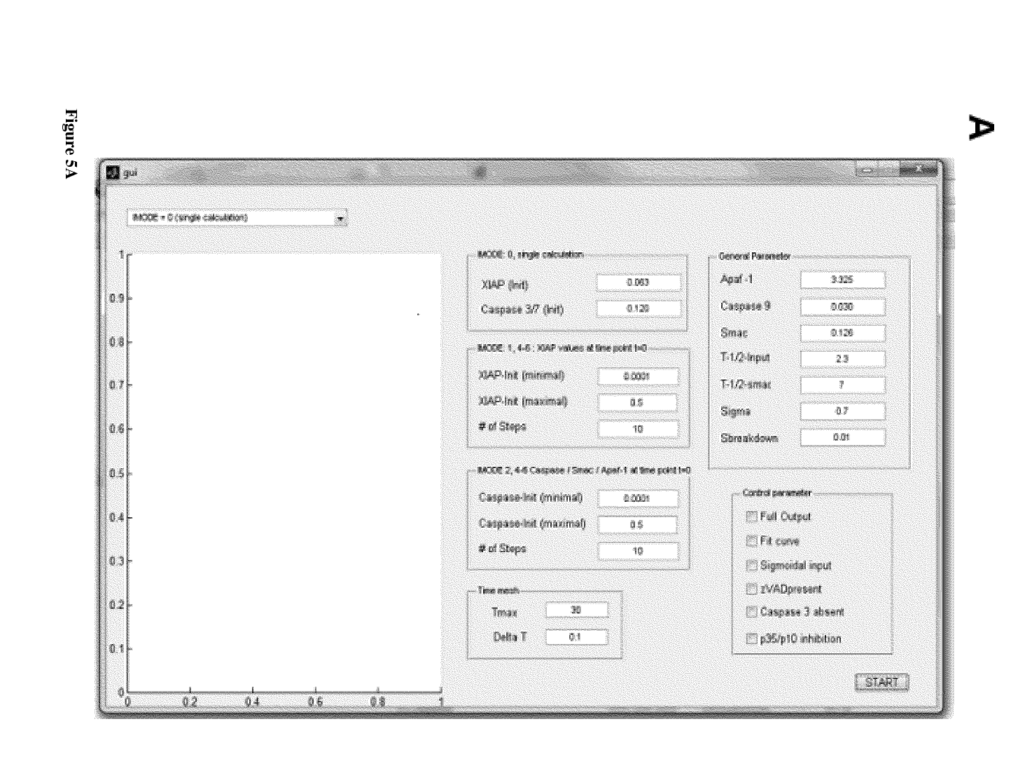

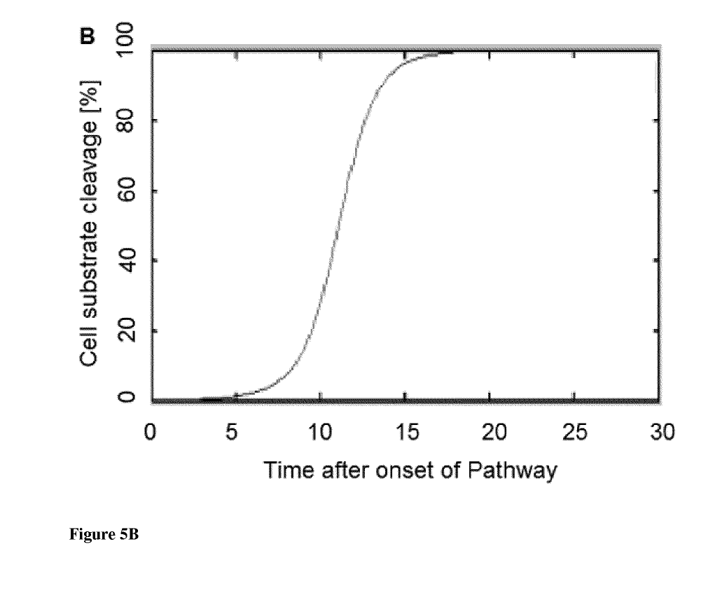

In a translational study (APO-COLON), the APOPTO-CELL model of the present invention was transferred into a clinical setting of colorectal cancer to predict response to chemotherapy from levels of apoptosis related proteins. Quantitative analysis of five pro-apoptotic or anti-apoptotic proteins in colorectal Dukes B (Stage II CRC) and Dukes C (Stage III CRC) patients was performed. The proteins analysed on quantitative to Western blot were Apaf-1, XIAP, pro-caspase 9, pro-caspase 3 and Smac (see FIG. 4). Actin was used to determine equal loading of the protein concentrations in the gel. In order to quantitate the concentration of the pro- and anti-apoptotic proteins (biomarkers) in the samples obtained from the patients, known protein concentration samples derived from HeLa cells are used as a standard protein concentration reference. These quantifications are necessary input for the APOPTO-CELL tool.

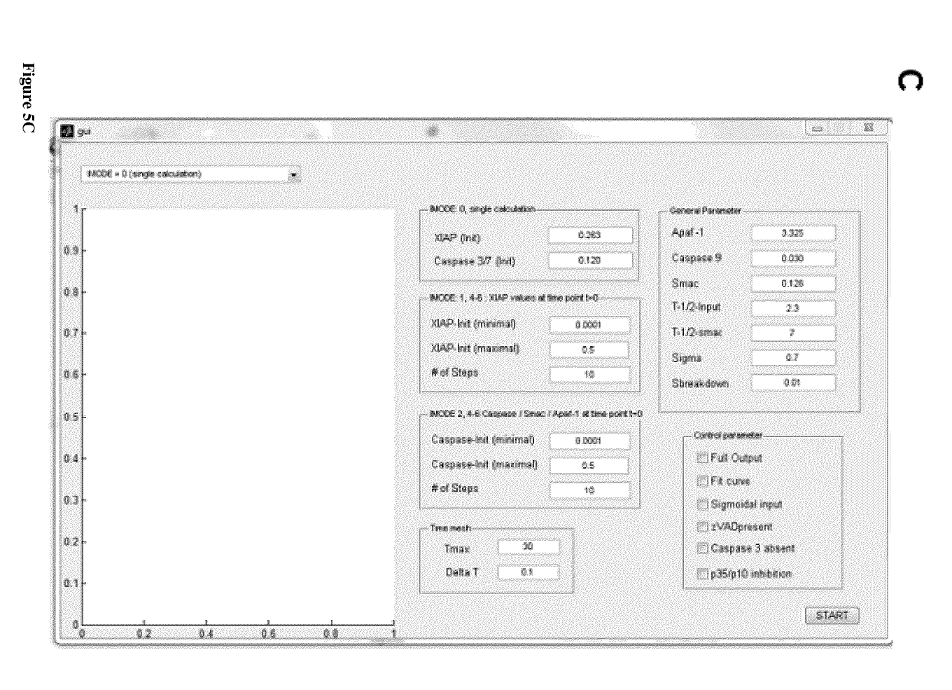

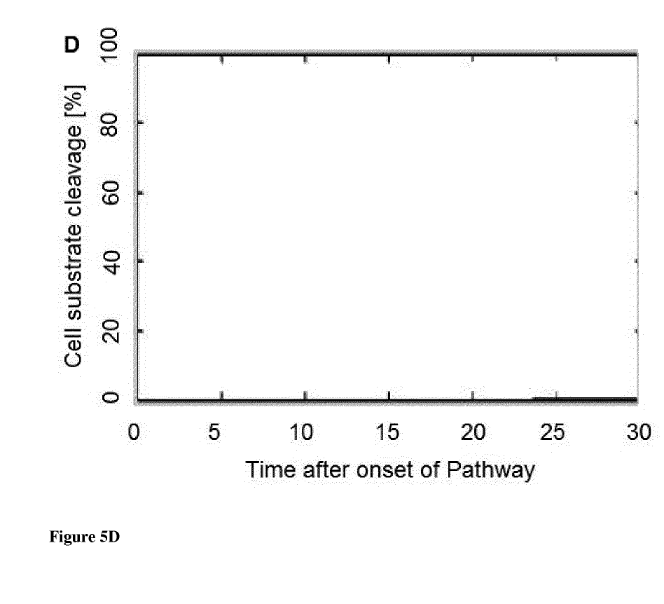

With the protein abundances quantified, the analysis is performed in the following way (see FIG. 5 for a pictorial representation of the interface):
I. Start the tool. The graphical user interface (GUI) will open.
II. Include the abundances of the five biomarkers into the GUI or use default values for any of them. The values shall be included in the input boxes besides the text ‘XIAP (int)’, ‘Caspase-3/7 (int)’, ‘APAF-1’, ‘Caspase 9’, ‘Smac’ for abundances in μM of proteins Xiap, Caspase-3, Apapf-1, caspase-9 and Smac, respectively.
III. Include a length of time that the protein-protein interaction will be calculated. Any value equal to or greater than 300 min can be chosen.
IV. Press start.
V. Wait till a new window opens. The graph displays the likelihood of cancer cells responding to the stimulus (and dying).
VI. Note the likelihood at maximum time point. This likelihood is positively correlated to a favourable clinical response.

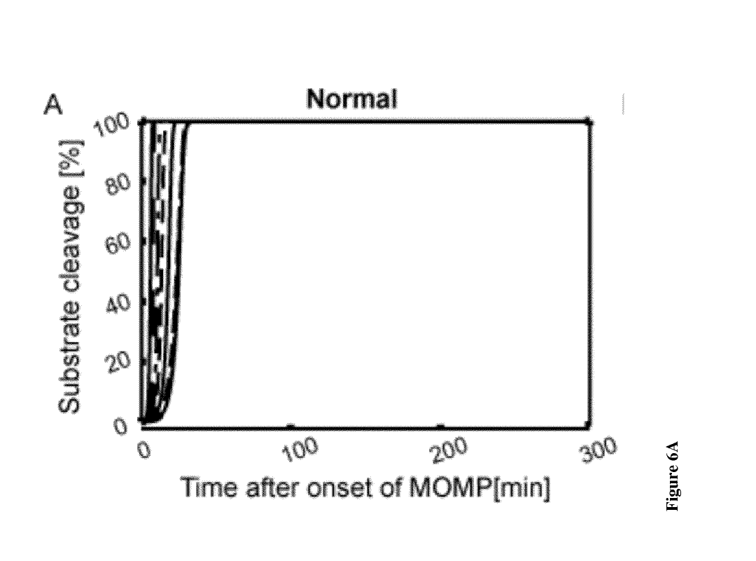

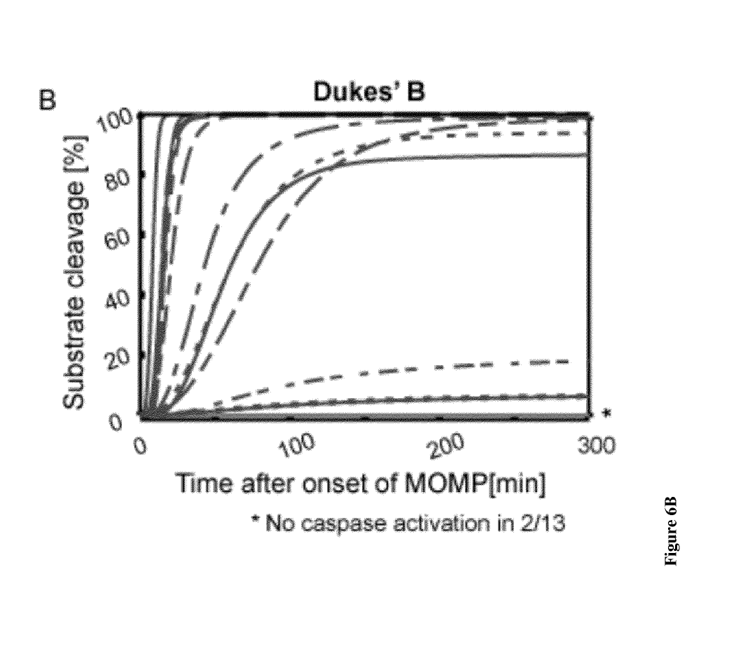

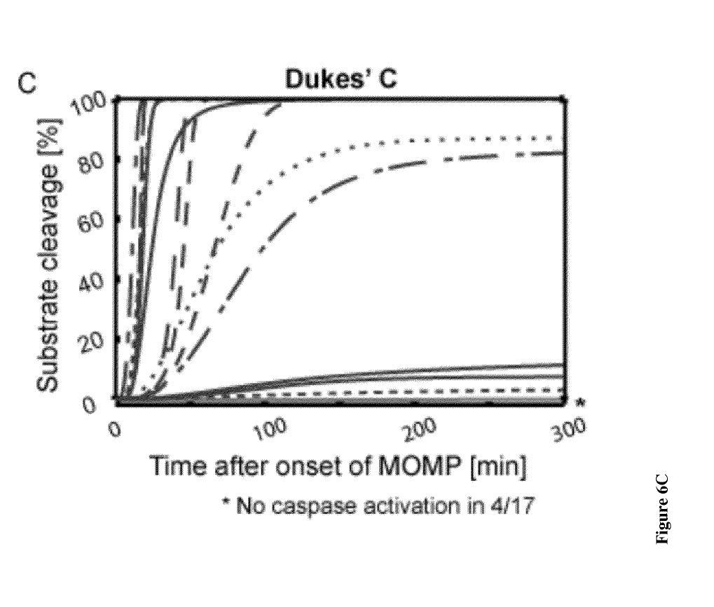

When the protein concentrations obtained from the results in FIG. 4 are inputted into the APOPTO-CELL computational model of the present invention (for example, as illustrated by the screen shots of the computational model in FIG. 5 and as explained above), the model predicts apoptosis impairment progressing with disease stage. For example, in normal tissue there is a prediction of 100% progression to apoptosis, a 69% probability of progression to apoptosis for Dukes' B stage patients analysed, and a 58% likelihood of progression to apoptosis for Dukes' C stage patients analysed (see FIG. 6). This data indicates that APOPTO-CELL is capable of resembling the clinically well-known fact that cell death is reduced during disease progression.

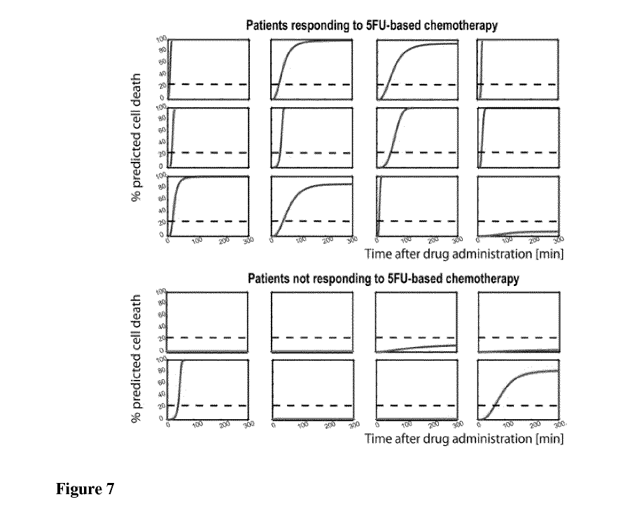

Of the 13 Dukes' B samples, 8 had a positive outcome and 5 had a negative outcome. 3 to of the positive outcome samples were from patients receiving chemotherapy treatment. Of the 17 Dukes' C samples, 9 had a positive outcome and 8 had a negative outcome. All of the Dukes' C samples were from patients receiving chemotherapy treatment. All 20 patients were receiving 5-FU/leucovorin, and of those 20 patients, two patients also received oxaliplatin and one also received irinotecan. To determine whether the computational model of mitochondrial apoptosis, APOPTO-CELL, of the present invention could predict treatment outcome in a clinical setting of patients treated for colorectal cancer, the data from those patients receiving chemotherapy were tested (see FIG. 7). Of the patients having positive [Upper panel: 12 patients] and negative [Lower panel: 8 patients] clinical responses to chemotherapy, the computational model of the present invention had an 85% correct prediction score (17 correct predictions from 20 known patient treatment outcomes). Caspase apoptosis was predicted in 11 of 12 colorectal cancer patients with positive outcome, while insufficient caspase activation was predicted in 6 of 8 patients with negative outcome. This good correlation between predicted treatment response and clinical response to chemotherapy will in future allow a priori prediction of whether a patient will or will not respond to classical FOLFOX and FOLFIRI-based chemotherapy. For the predicted non-responder group, co-treatment regimes will be applied, as illustrated in the flow-chart of FIG. 3. This clearly demonstrates how APOPTO-CELL can be employed to predict personalized patient response to distinct adjuvant therapies.

Furthermore, the APOPTO-CELL model of the present invention significantly outscores discriminant statistical analysis using the same data set as exemplified by two statistical approaches, discriminant analysis and principal component analysis. None of the plasticising approaches were able to identify any combination of APAF-1, XIAP, SMAC and Pro-Caspase-3 and -9 that may act as a predictor of clinical treatment response. Discriminant analysis was able to correctly classify 75% (15/20) of patients, but did not reach statistical significance, exhibiting a P value of 0.109. Likewise, principal component analysis did not give any linear combination of protein concentrations that was able to distinguish between clinical responders and non-responders at a significance level of p<=0.05.

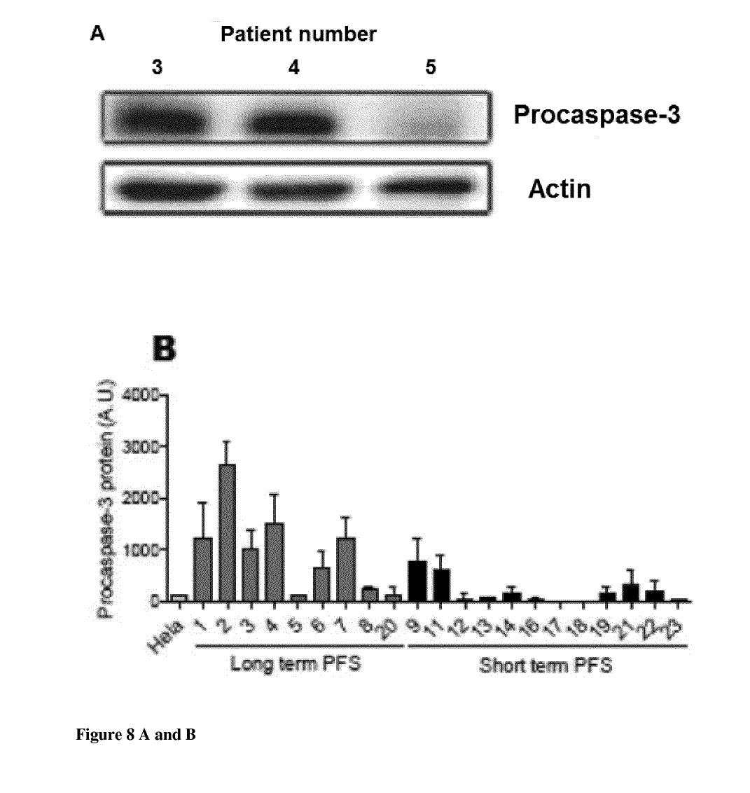

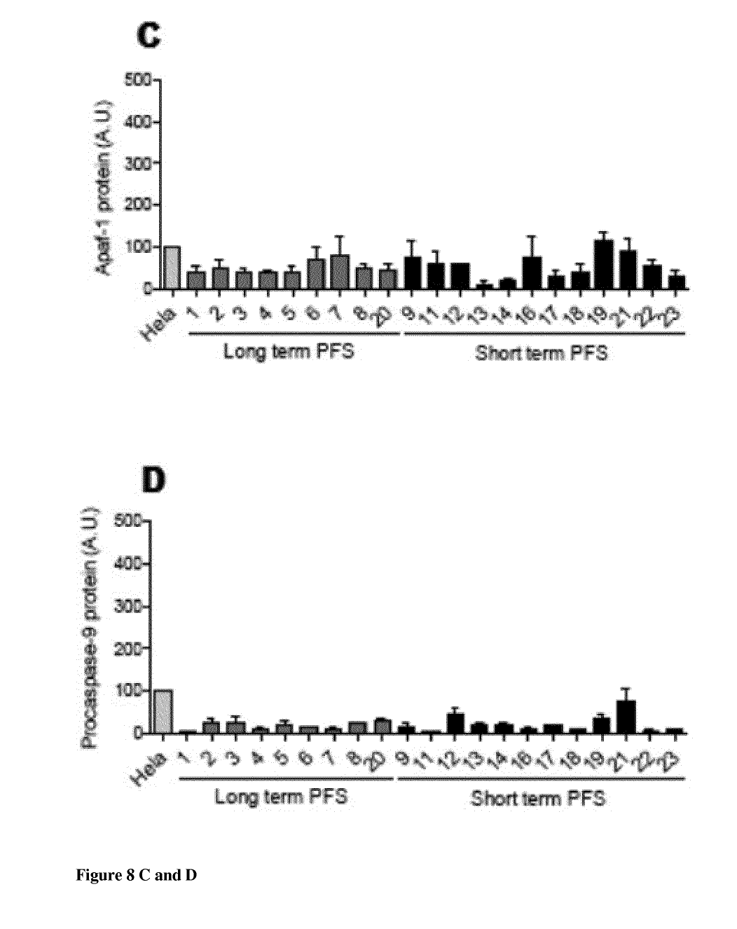

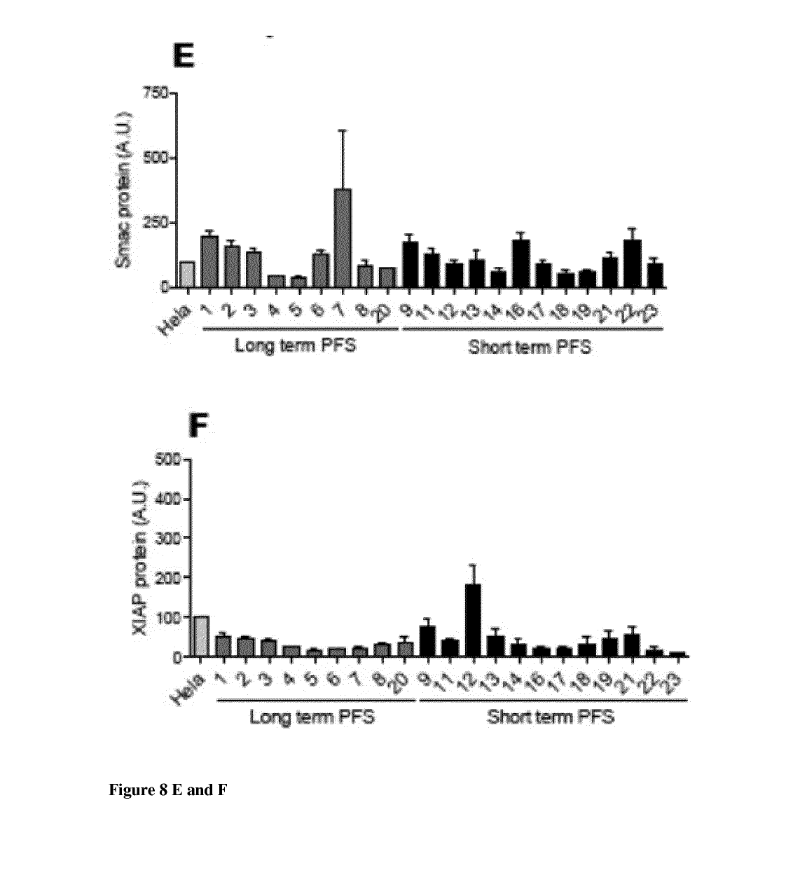

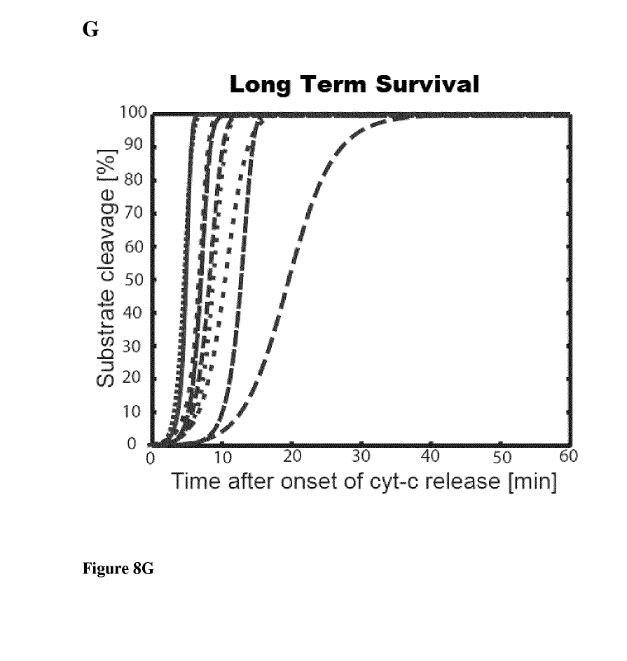

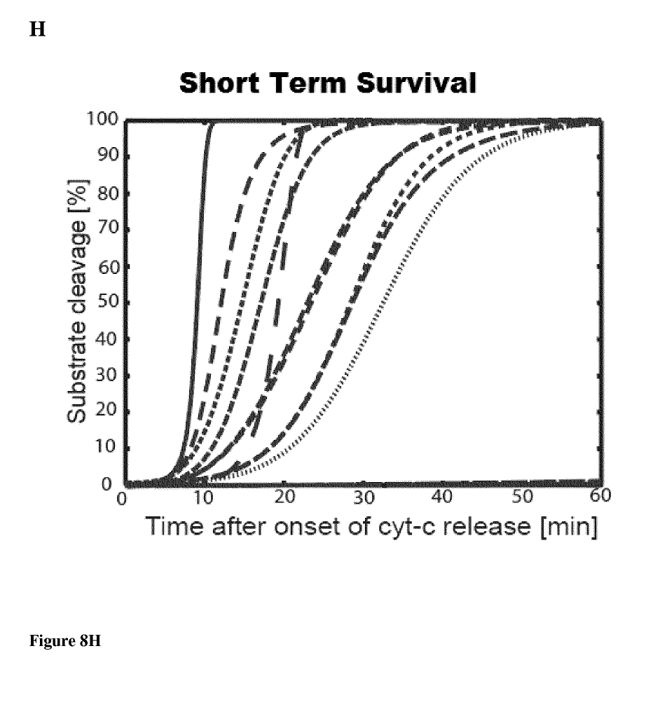

FIG. 8 illustrates that the APOPTO-CELL tool of the present invention predicts correlation between the likelihood of cancer cells undergoing apoptosis with survival for patients suffering from glioblastoma multiformae (GBM). In panels A-F, protein quantifications (Panel A) from tumour samples of GBM patients were quantified by quantitative Western Blotting. Protein levels of the APOPTO-CELL proteins Procaspase-3 (B), APAF-1 (C), Procaspase-9 (D), Smac (E), and XIAP (F) were quantified. The prediction of the susceptibility of cancer cells undergoing cell death (apoptosis) is clearly illustrated in panels G and H to correlate with duration of patient survival. 20 patients were grouped into either (G) Long term or (H) short term survivors according their clinical outcome. Patients that survived longer were also predicted to have tumours that are more susceptible to cancer cell death indicated by shorter response time to treatment (curves reach 100% more quickly).

Therefore, tools such as the present invention that not only includes qualitative, but also quantitative protein expression and information on the protein-protein network of apoptosis-regulating proteins can be employed for a better prediction of patient response to therapy. Moreover, a model such as the present invention that studies a (mathematically non-linear) protein-protein network is superior to (mathematically linear) statistical approaches.

This protein-protein interaction network analysed in APOPTO-CELL is assumed to be initiated by DNA damaging agent-based chemotherapy. Based on this individual patient data, likelihood for the success of treatment for this patient will be calculated. If this likelihood of response is low, the tool allows the inclusion of additional co-treatment regimes that affect the same protein-protein pathways in order to increase tumour cell death. Several co-treatment regimes such as treatments based on proteasome inhibitors, activators of cell death proteases or mimetics of other cell death activating proteins are illustrated in FIG. 9.

### Tables

TABLE 1
Abbreviations of proteins in the mathematical model.

| Number | Abbreviation | Explanation |
|---|---|---|
| 1 | Casp-9 | Apoptosome associated caspase-9 (p35/p12) |
| 2 | Casp-9P | Apoptosome associated caspase-9 processed (p35/p10) |
| 3 | Procasp-3 | Procaspase-3 |
| 4 | Casp-3 | Caspase-3 |
| 5 | XIAP | Free x-linked Inhibitor of Apoptosis Protein |
| 6 | XIAP~Casp-9 | XIAP in complex with caspase-9 (p35/p12) |
| 7 | XIAP~Casp-3 | XIAP in complex with caspase-3 |
| 8 | XIAP~Casp-3~Casp-9 | XIAP in complex with both caspase-3 and caspase-9 (p35/p12) |
| | | (not considered in final calculation) |
| 9 | Bir12 | XIAP fragment comprising baculoviral IAP repeats 1 and 2 |
| 10 | Bir3R | XIAP fragment comprising baculoviral IAP repeat 3 and RING domain |
| 11 | Bir12~Casp-3 | XIAP Bir12 fragment in complex with caspase-3 |
| 12 | Bir3R~Casp-9 | XIAP Bir3R fragment in complex with caspase-9 (p35/12) |
| 13 | XIAP~p2-frag | XIAP in complex with the caspase-9 (p35/p12) derived p2 fragment |
| 14 | Smac | Second mitochondria-derived activator of caspases |
| 15 | 2Smac~XIAP | Smac dimer in complex with XIAP |
| 16 | XIAP~Casp-3~p2-fragment | XIAP in complex with both caspase-3 and the p2 fragment |
| 17 | XIAP~{p2-frag}~2Smac | XIAP in complex with SMAC dimer and p2 fragment |
| 18 | Smac~Bir12 | Smac in complex with the XIAP Bir12 fragment |
| 19 | Smac~Bir3R | Smac in complex with the XIAP Bir3R fragment |
| 20 | Substrate | DEVD effector caspase substrate |

TABLE 2
Input function parameters and initial protein concentrations

| | Half time | |
|---|---|---|
| Mitochondrial release | [min] | References |
| Cytochrome c release | 1.5 | (Goldstein J C 2000; |
| kinetic (t_{Cyt-c1/2}) | | Rehm M 2003) |
| Apoptosome formation | 2.3 | This study |
| kinetic (t_{Apop 1/2}) | | (Supplementary |
| | | Information 2) (Hill, |
| | | Adrain et al. 2004; |
| | | Twiddy, Brown et al. |
| | | 2004) |
| Smac release kinetic | 7 | (Rehm M 2003) |
| (t_{Smac, 1/2}) | | |
| | Initial | |
| Initial protein | concentration | |
| concentrations | [μM] | |
| Apaf-1 | 0.372 | This study |
| Cytochrome c | 10 | (Waterhouse, |
| | | Goldstein et al. 2001) |
| Procaspase-9 | 0.03 | This study |
| Procaspase-3 | 0.12 | This study |
| XIAP | 0.063 | This study |
| Smac | 0.126 | Estimated to be |
| | | twice the XIAP conc. |

TABLE 3
The modelled reaction network

| # | | | | Reaction | | |
|---|---|---|---|---|---|---|
| 1 | Procasp-3 | | → | Production | (v = kₒₙ − k_{off}*[c]) | |
| 2 | XIAP | | → | Production | (v = kₒₙ − k_{off}*[c]) | |
| 3 | Casp-9 | + Procasp-3 | → | Casp-9 | + Casp-3 | |
| 4 | Casp-9 | + Casp-3 | → | Casp-9P | + Casp-3 | |
| 5 | Casp-9P | + Procasp-3 | → | Casp-9P | + Casp-3 | |
| 6 | Procasp-3 | + Casp-3 | → | Casp-3 | + Casp-3 | |
| 7 | Casp-3 | + XIAP | [symbol] | XIAP~Casp-3 | | |
| 8 | Casp-3 | + XIAP~Casp-9 | [symbol] | XIAP~Casp-9~Casp-3 | | |
| 9 | Casp-3 | + XIAP~{p2-frag} | [symbol] | XIAP~{p2-frag}~Casp-3 | | |
| 10 | Casp-3 | + Bir12 | [symbol] | Bir12~Casp-3 | | |
| 11 | Casp-3 | + XIAP | → | Bir12 | + Bir3R | + Casp-3 |
| 12 | Casp-3 | + XIAP~Casp-9 | → | Bir12 | + Bir3R~Casp-9 | + Casp-3 |
| 13 | Casp-3 | + XIAP~Casp-3 | → | Casp-3 | + Bir12~Casp-3 | + Bir3R |
| 14 | Casp-3 | + XIAP~{p2-frag} | → | Casp-3 | + Bir12 | + Bir3R~{p2-frag} |
| 15 | Casp-3 | + XIAP~{p2-frag}~Casp-3 | → | Casp-3 | + Bir12~Casp-3 | + Bir3R~{p2-frag} |
| 16 | Casp-3 | + XIAP~Casp-9~Casp-3 | → | Casp-3 | + Bir12~Casp-3 | + Bir3R~Casp-9 |
| 17 | Casp-3 | + XIAP~2Smac | → | Casp-3 | + Bir12~Smac | + Bir3R~Smac |
| 18 | Casp-3 | + XIAP~Casp-9~Casp-3 | → | Casp-3 | + Casp-9P | + XIAP~{p2-frag}~Casp-3 |
| 19 | Casp-3 | + XIAP~Casp-9 | → | Casp-3 | + Casp-9P | + XIAP~{p2-frag} |
| 20 | Casp-3 | + Bir3R~Casp-9 | → | Casp-3 | + Bir3R~{p2-frag) | + Casp-9P |
| 21 | Casp-9 | + XIAP | [symbol] | XIAP~Casp-9 | | |
| 22 | Casp-9 | + XIAP~Casp-3 | [symbol] | XIAP~Casp-9~Casp-3 | | |
| 23 | Casp-9 | + Bir3R | [symbol] | Bir3R~Casp-9 | | |
| 24 | Bir3R~{p2-frag} | | → | Bir3R | | |
| 25 | XIAP~{p2-frag} | | → | XIAP | | |
| 26 | XIAP | + 2 Smac | [symbol] | XIAP~2Smac | | |
| 27 | XIAP~Casp-9 | + 2 Smac | [symbol] | XIAP~2Smac | + Casp-9 | |
| 28 | XIAP~Casp-3 | + 2 Smac | [symbol] | XIAP~2Smac | + Casp-3 | |
| 29 | XIAP~Casp-9~Casp-3 | + 2 Smac | [symbol] | XIAP~2Smac | + Casp-3 | + Casp-9P |
| 30 | Bir12 | + Smac | [symbol] | Bir12~Smac | | |
| 31 | Bir3R | + Smac | [symbol] | Bir3R~Smac | | |
| 32 | Bir12~Casp-3 | + Smac | [symbol] | Bir12~Smac | + Casp-3 | |
| 33 | Bir3R~Casp-9 | + Smac | [symbol] | Bir3R~Smac | + Casp-9 | |
| 34 | XIAP~{p2-frag} | + 2Smac | [symbol] | XIAP~{p2-frag}~2Smac | | |
| 35 | | Casp-9P | → | Degradation | | |
| 36 | | Casp-9 | → | Degradation | | |
| 37 | | Casp-3 | → | Degradation | | |
| 38 | | XIAP~Casp-3 | → | Degradation | | |
| 39 | | XIAP~Casp-9~Casp-3 | → | Degradation | | |
| 40 | | XIAP~Casp-9 | → | Degradation | | |
| 41 | | XIAP~{p2-frag} | → | Degradation | | |
| 42 | | XIAP~{p2-frag}~Casp-3 | → | Degradation | | |
| 43 | | XIAP~{p2-frag}~Smac | → | Degradation | | |
| 44 | | XIAP~2Smac | → | Degradation | | |
| 45 | | Bir12 | → | Degradation | | |
| 46 | | Bir3R | → | Degradation | | |
| 47 | | Bir12~Smac | → | Degradation | | |
| 48 | | Bir3R~Smac | → | Degradation | | |
| 49 | | Bir12~Casp-3 | → | Degradation | | |
| 50 | Bir3R~Casp-9 | | → | Degradation | | |
| 51 | Bir3R~{p2-frag} | | → | Degradation | | |
| 52 | Smac | | → | Degradation | | |
| 53 | Casp-3 | + Substrate | → | Casp-3 | + Cleavage products | |

TABLE 4
Kinetic constants and binding constants used in the model

| Parameter | k_{cat} ^{a)} | kₒₙ ^{a)} | k_{off} ^{b)} | | Ref. | Reaction(s) |
|---|---|---|---|---|---|---|
| Caspase-3 protein production rate | — | kₒₙ * XIAPᵢₙᵢₜ | 0.0039 | (6.5E−5) | (Eissing T 2004) | 1 |
| XIAP protein production rate | — | kₒₙ * XIAPᵢₙᵢₜ | 0.0116 | (1.9E−4) | (Eissing T 2004) | 2 |

| | | | | | | |
|---|---|---|---|---|---|---|
| Caspase-9 (p35/p12) activity | 6 | (1.0E+5) | — | — | (Garcia-Calvo M 1998) | 3 |
| Caspase-3 activity | 12 | (2.0E+5) | — | — | (Stennicke H R 2000) | 4, 11-20, 53 |
| Caspase-9 (p35/p10) activity | 48 | (8.0E+5) | — | — | (Zou H 2003) | 5 |
| Caspase-3 self processing activity | 2.4 | (4.0E+4) | — | — | (Zou H 2003) | 6 |

| XIAP or Bir12 binding to caspase-3 | — | 156 | (2.6E+6) | 0.144 | (2.4E−3) | (Riedl S J 2001) | 7-10 |
|---|---|---|---|---|---|---|---|
| XIAP or Bir3R binding to caspase-9 | — | 156 | (2.6E+6) | 0.144 | (2.4E−3) | (Deveraux, Leo et | 21-23 |
| (p35/p12) | | | | | | al. 1999; Sun, Cai | |
| | | | | | | et al. 2000; Riedl | |
| | | | | | | S J 2001) | |
| Degradation of p2, XIAP or BIR3R | — | — | | — | | n.a. | 24, 25 |
| liberation | | | | | | | |
| Smac binding to XIAP | — | 420 ^{d)} | (7E+12) | 0.133 | (2.22E−3) | (Huang Y 2003) | 26 |
| Smac binding to XIAP (concurring with | — | 420 ^{d)} | (7E+12) | 156 ^{a)} | (2.6E+6) | (Huang Y 2003), ^{e)} | 27-29, 34 |
| caspases) | | | | | | | |
| Smac binding to Bir12 | — | 4.45 | (7.4E+4) | 31.9 | (5.32E−1) | (Huang Y 2003) | 30 |
| Smac binding to Bir3R | — | 0.33 | (5.5E+3) | 14.2 | (2.37E−1) | (Huang Y 2003) | 31 |
| Smac-BIR12 in concurrence to caspase-3 | — | 4.45 | (7.4E+4) | 156 ^{a)} | (2.6E+6) | (Huang Y 2003), ^{e)} | 32 |
| Smac-BIR3R in concurrence to caspase-9 | — | 0.33 | (5.5E+3) | 156 ^{a)} | (2.6E+6) | (Huang Y 2003), ^{e)} | 33 |

| | | | | | | |
|---|---|---|---|---|---|---|
| General protein degradation rate | — | 0.0058 ^{b)} | (9.67E−5) | — | (Eissing T 2004) | 35-37, 41, 45, |
| | | | | | | 47, 49, 52 |
| XIAP- or Bir3R- enforced degradation | — | 0.0347 ^{b)} | (5.78E−4) | — | (Yoo S J, Huh et | 38-40, 42, 43, |
| rate | | | | | al. 2002) | 44, 46, 48, 50-51 |

Units:
^{a)} μM⁻¹ min⁻¹ (M⁻¹ s⁻¹),
^{b)} min⁻¹ s⁻¹),
c) μM min⁻¹(M s⁻¹),
^{d)} μM⁻² min⁻¹(M⁻² s⁻¹)
^{e)} Backward reaction remodelled from forward reaction of concurring reaction (i.e. caspase - XIAP interaction)

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an to electrical or an optical cable or by radio or other means.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

### REFERENCES

Chu Liang-Hui et al. (2008). “Construction of a cancer-perturbed protein-protein interaction network for discovery of apoptosis drug targets.” BMC System Biology, vol. 2(1): 56.
Deveraux, Q. L., E. Leo, et al. (1999). “Cleavage of human inhibitor of apoptosis protein XIAP results in fragments with distinct specificities for caspases.” Embo J 18(19): 5242-5251.
Eissing T, C. H., Gilles E D, Allgoewer F, Bullinger E, Scheurich P (2004). “Bistability analyses of a caspase activation model for receptor induced apoptosis*.” J. Biol. Chem. 279(35): 36892-36897.
Garcia-Calvo M, P. E., Leiting B, Ruel R, Nicholson D W, Thornberry N A (1998). “Inhibition of human caspases by peptide-based and macromolecular inhibitors.” J. Biol. Chem. 273(49): 32608-32613.
Huang Y, R. R., Myszka D G, Wu H (2003). “Requirement of both the second and third BIR domains for the relief of X-linked inhibitor of apoptosis protein (XIAP)-mediated caspase inhibition by Smac.” J. Biol. Chem. 278(49): 49517-49522.
Riedl S J (2001). “Structural Basis for the Inhibition of Caspase-3 by XIAP.” Cell 104: 791-800.
Stennicke H R, R. M., Meldal M, Salvesen G S (2000). “Internally quenched fluorescent peptide substrates disclose the subsite preferences of human caspases 1, 3, 6, 7 and 8.” Biochem. J. 350: 563-568.
Sun, C., M. Cai, et al. (2000). “NMR structure and mutagenesis of the third Bir domain of the inhibitor of apoptosis protein XIAP.” J Biol Chem 275(43): 33777-33781.
Yoo S J, J. R. Huh, et al. (2002). “Hid, Rpr and Grim negatively regulate DIAP1 levels through distinct mechanisms.” Nat Cell Biol 4(6): 416-424.
Zou H, Y. R., Hao J, Wang J, Sun C, Fesik S W, Wu J C, Tomaselli K J, Armstrong R C (2003). “Regulation of the Apaf-1/caspase-9 apoptosome by caspase-3 and to XIAP.” J. Biol. Chem. 278(10): 8091-8098.

1. A computer-implemented method for predicting an individual's response to a treatment for cancer, the method comprising:
assaying a biological sample from the individual to determine abundance values for a panel of two or more biomarkers comprising pro-apoptotic and anti-apoptotic biomarkers;
providing a computer system having at least one processor and associated memory; the computer system including an ordinary differential equation-based mathematical non-linear protein-protein network computational model of a mitochondrial apoptosis pathway;
inputting the abundance values for the two or more biomarkers into the ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and
processing said abundance values using said computational model to produce a value to predict the individual's response to treatment.

2-26. (canceled)

27. A computer-implemented method according to claim 1 wherein the processing step comprises inputting the abundance values into the computational model to provide a (caspase) apoptosis status, and correlating said status with a known response to provide said value to predict the individual's response to treatment.

28. A computer-implemented method according to claim 1, wherein said processing step calculates resulting effector caspase activation profile over time and compares the result with known results to provide said value to predict the individual's response to treatment.

29. A computer-implemented method according to claim 1, wherein the value to predict the individual's response is calculated using ordinary differential equations defined by
$$\frac{d {c}_{i}}{d t} = {\sum }_{j} {S}_{ij} * {v}_{j} + \frac{d {F}_{i}}{d t} , where \frac{d {c}_{i}}{d t}$$
represents the concentration change of molecule i over time, the velocity vⱼ is the reaction rate of reaction j, Sᵢⱼ denotes the stoichiometric matrix linking the reaction rates to the affected molecules, and
$$\frac{d {F}_{i}}{d t}$$
denotes a rate factor describing an external flux balance of the substance i.

30. A computer-implemented method according to any one of claim 1, wherein the value to predict the individual's response is calculated using ordinary differential equations defined by
$$\frac{d {c}_{i}}{d t} = {\sum }_{j} {S}_{ij} * {v}_{j} + \frac{d {F}_{i}}{d t} , where \frac{d {c}_{i}}{d t}$$
represents the concentration change of molecule i over time, the velocity vⱼ is the reaction rate of reaction j, Sᵢⱼ denotes the stoichiometric matrix linking the reaction rates to the affected molecules, and
$$\frac{d {F}_{i}}{d t}$$
denotes a rate factor describing an external flux balance of the substance i and wherein the reaction rates are proportional to the product of the concentrations of the reacting substances.

31. A computer-implemented method according to any claim 1, wherein the panel of biomarkers is selected from Apaf-1, procaspase-9, procaspase-3, XIAP and Smac.

32. A computer-implemented method according to claim 1, wherein the abundance value for each biomarker is representative of protein levels for the sample.

33. A computer-implemented method according to claim 1 wherein the cancer is selected from the group consisting of myeloma, prostate cancer, glioblastoma, lymphoma, fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

34. A computer-implemented method according to claim 1 in which the panel of biomarkers comprises Smac and Apaf-1, and optionally one or more biomarkers selected from procaspase-9, procaspase-3 and XIAP.

35. A computer-implemented method according to claim 1 in which the panel of biomarkers comprises Smac, and one or more biomarkers selected from procaspase-9, procaspase-3 and XIAP.

36. A computer-implemented method according to claim 1 in which the panel of biomarkers comprises Apaf-1, and one or more biomarkers selected from procaspase-9, procaspase-3 and XIAP.

37. A computer-implemented system for predicting an individual's response to a treatment for cancer, the system comprising:
a computer system having at least one processor and associated memory; the computer system including an ordinary differential equation-based mathematical non-linear protein-protein network computational model of a mitochondrial apoptosis pathway and being adapted to produce a value to predict the individual's response to treatment as a function of two or more abundance values;
means for assaying a biological sample from the individual to determine the abundance values for a panel of two or more biomarkers comprising pro-apoptotic and anti-apoptotic biomarkers;
the computer system being adapted to receive the abundance values for the two or more biomarkers into an ordinary differential equation-based mathematical non-linear protein-protein network computational model of a mitochondrial apoptosis pathway, wherein the pathway is activated through the permeabilisation of a cell's outer mitochondrial membrane by chemotherapeutically-induced stress; and for processing said abundance values using said computational model to provide a value to predict the individual's response to treatment.

38. A computer-implemented system according to claim 37, wherein the processing comprises inputting the abundance values into the computational model to provide a (caspase) apoptosis status, and correlating said status with a known response to provide said value to predict the individual's response to treatment.

39. A computer-implemented system according to claim 37, wherein said processing means calculates resulting effector caspase activation profile over time and compares the result with known results to provide said value to predict the individual's response to treatment.

40. A computer-implemented system according to claim 37, wherein the panel of biomarkers is selected from Apaf-1, procaspase-9, procaspase-3, XIAP and Smac.

41. A computer-implemented system according to claim 37, wherein the abundance value for each biomarker is representative of protein levels for the sample.

42. A computer-implemented system according to claim 37 wherein the means of determining the abundance of the panel of biomarkers further comprises a means for obtaining protein profiles by any one or more of: tissue microarray immunostaining, immunohistochemistry, reverse phase protein array analysis, or quantitative Western blot.

43. A computer-implemented system according to claim 37 wherein the cancer is selected from the group consisting of myeloma, prostate cancer, glioblastoma, lymphoma, fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

44. A method of identifying an individual having cancer who is suited for treatment with a chemotherapeutic agent, which method employs a step of identifying an individual who will respond to a treatment according to the method of claim 1, wherein the individual identified is treated with the chemotherapeutic agent.
